(19) United States
(12) Patent Application Publication
(10) Pub. No.: US 2004/0214242 A1
(43) Pub. Date: Oct. 28, 2004

(54) Method for the isolation of large variances of specific molecules for a target molecule from phagemid gene libraries, and a test kit

(75) Inventors: Steffen Goletz, Berlin (DE); Peter Astrup Christensen, Berlin (DE); Peter Kristensen, Tranbjerg J (DK)

Correspondence Address: BRUCE LONDA, NORRIS, MCLAUGHLIN & MARCUS, P.A., 220 EAST 42ND STREET, 30TH FLOOR, NEW YORK, NY, 10017, US

(73) Assignee: NEMOD Immuntherapie AG, Berlin (DE)

(21) Appl. No.: 10/198,472

(22) Filed: Jul. 18, 2002

(30) Foreign Application Priority Data
Jul. 18, 2001 (DE) 101 35 039.2

(51) Int. Cl. C12Q 1/70; G01N 33/567; C12P 21/06; C12P 21/02; C12N 5/06; C07K 16/00

(52) U.S. Cl. 435/7.21; 435/5; 435/69.1; 435/320.1; 435/326; 530/388.1

(57) ABSTRACT

The invention relates to a method for the isolation of large variances of specific molecules for a target molecule from phagemid gene libraries and to a test kit, said method being performed by starting with a phagemid gene library including large variances of a molecule or large variances of portions of a molecule which, in the meaning of the invention, acts as a specific molecule, directly, or via a linker, coupled covalently to the phage coat protein pIII of the filamentous phage M13 as a fusion protein on the surface of phages, incubating the library phages including the phages expressing the fusion protein with a target molecule, eluting the phages specifically bound to the target molecule via the fusion protein with a selection molecule which terminates binding between the specific molecule and the target molecule and binds to the target molecule, inactivating the eluted phages failing to express the fusion protein in a way so as to render them incapable of infecting bacteria, using the eluted phages expressing the fusion protein to infect bacteria, and determining and obtaining the variances of the specific molecule from the infected clones.

The invention relates to an efficient method for the isolation of large variances of specific molecules for a target molecule from phagemid gene libraries, which method allows for the isolation of large variances of specific molecules, e.g. antibody fragments such as Fab fragments or scFv fragments, with low work input. In a special embodiment of the invention, the specific molecules to be isolated are surrogate molecules for the selection molecule employed in the method according to the invention.

The invention is also directed to a test kit used to perform the method according to the invention.

The phage display technique is known to be highly suitable e.g. for the rapid production of specific recombinant human antibody fragments. To produce Fab fragments or single-chain Fv antibody fragments (scFv) with the desired specificity, they are selected from antibody libraries including the appropriate antigens. However, these antigens are not always available in their pure forms; rather, they may be part of an antigen mixture, for example. Even pure antigens do consist of different epitopes, and phage display selections, as a rule, in by far the most cases produce antibody fragments against one or just a few dominant epitopes of the antigen. The reason for this is that, as a rule, several selection cycles are required to accumulate and isolate specific antibody fragments. Eventually, antibody fragments binding with highest affinity, or those phages having a growth advantage during amplification between the selection cycles, or those having a combination of growth advantage and high affinity will be obtained. As a rule, antibody fragments having lower affinities to epitopes of the target molecule or those suffering from a growth disadvantage in the phage display system will not be selected. This effect increases with increasing number of selection cycles, resulting in a loss of variance in potential binding species during sequential selection cycles and impeding selection of lower affinity antibodies or those against minor components of antigen mixtures or cell surfaces.

A selection method is known from WO 99/58655 wherein selection of the phages expressing the desired fusion protein, e.g. the antibody fragment, coupled to pIII is performed using the helper phage KM13 which encodes a modified pIII protein having a proteolytic cleavage site. In contrast, the pIII portion of the fusion protein cannot be cleaved by proteolysis, so that the infectivity of phages expressing the fusion protein is retained subsequent to the proteolytic treatment.

The above selection method allows for elimination of phages failing to express the desired fusion protein, which phages frequently give rise to a massive background in phage display systems. Using this method, it is possible to obtain specific molecules in a more facile fashion. However, this selection method does not permit isolation of large variances of specific molecules such as antibody fragments.

It was the object of the invention to provide a selection method for the isolation of specific molecules from phagemid gene libraries, which method would enable providing large variances of molecules specific to a target molecule, particularly antibody fragments. More specifically, the production of surrogate molecules such as scFv antibody fragments using the standard method of hybridoma technique is difficult and time-consuming and therefore, the method of the invention is intended to provide the above-mentioned antibody fragments in an effective manner.

The object of the invention is accomplished by starting with a phagemid gene library including large variances of a molecule or large variances of portions of a molecule which, in the meaning of the invention, acts as a specific molecule, directly, or via a linker, coupled covalently to the phage coat protein pIII of the filamentous phage M13 as a fusion protein on the surface of phages, incubating the library phages including the phages expressing the fusion protein with a target molecule, eluting the phages specifically bound to the target molecule via the fusion protein with a selection molecule which terminates binding between the specific molecule and the target molecule and binds to the target molecule, inactivating the eluted phages failing to express the fusion protein in a way so as to render them incapable of infecting bacteria, using the eluted phages expressing the fusion protein to infect bacteria, and determining and obtaining the variances of the specific molecule from the infected clones.

According to the invention, inactivation of the infectivity of phages not bearing any specific molecule can also be performed so as not to represent the last step but rather, as early as prior to incubation with target molecule and specific elution.

The phagemid gene libraries of the filamentous M13 phage which are used to perform the method and express large variances of the desired molecules can be furnished according to per se known methods (Nissim, A. et al., 1994, EMBO J. 13, 692-69; Griffiths, A. et al., 1994, EMBO 25 J. 13, 3245-3260; http://www.mrc-cpe.cam.ac.uk/˜phage/). Furnishing such libraries is well-known to those skilled in the art.

In a preferred embodiment of the invention, antibody fragments are isolated as specific molecules, preferably single-domain fragments, Fab or scFv fragments, or carrier proteins with one or more variable portions, preferably those molecules which have a basic structure permitting specific interaction of the variable portions with the target molecule, or peptides having completely or partially variable portions. Carrier proteins are understood to be those protein structures serving as framework/basic structure to present the variable portions but also, protein structures in general. Variable portions can be situated within the carrier protein structure, they can be part of the carrier protein structure or located at the extremities of the carrier proteins.

In the first step of the inventive method, phages bearing the desired specific molecules are selected in a per se conventional fashion by incubating with a target molecule, said target molecule being used immobilized on a solid phase such as magnetic beads or plastic surfaces (phage panning).

In the second step of the method according to the invention, the phages specifically bound to the target molecule via the fusion protein are eluted with a selection molecule which terminates the linkage between the specific molecule and the target molecule (and binds to the target molecule). In a preferred fashion, the selection molecule terminates the linkage between the specific molecule and the target molecule e.g. by having a competitive or allosteric effect.

In a preferred embodiment of the invention, antibodies or antibody fragments specifically binding an antigen are used as target molecules. The antigen or suitable portions thereof are used as selection molecule so as to obtain large variances of specific molecules representing surrogate molecules for the antigen. The antigen or portions thereof can be used as such or coupled to carrier structures.

In another preferred embodiment of the invention, receptors or portions of receptors specifically binding a ligand are used as target molecules. The ligand or suitable portions of the ligand are used as selection molecule so as to obtain large variances of specific molecules representing surrogate molecules for the ligand. The ligands or portions thereof can be used as such or coupled to carrier structures.

In a further preferred embodiment of the invention, molecules of biological or non-biological origin are used as target molecules, which neither are antibodies nor receptors in the above-mentioned implication, but specifically bind one or more molecules from which surrogate molecules are to be produced. One representative example are lectins. Molecules or corresponding portions of molecules binding the target molecule, thus eluting the specific molecules, are used as selection molecule so as to obtain large variances of specific molecules. When using lectins as target molecule according to the invention, carbohydrate structures or portions thereof serve as selection molecule, and these carbohydrate structures can be used as such or coupled to carriers.

In the third step of the method according to the invention, the eluted phages failing to express the fusion protein are inactivated in a way so as to be incapable of infecting bacteria. As described above, this step might also be performed as early as prior to incubation with target molecule and therefore would not take place at this point. The eluted phages expressing the fusion protein are used to infect bacteria. By virtue of a resistance gene encoded in the genetic material of the phagemid, bacteria infected by a phage and thus bearing the gene for the corresponding specific molecule are preferably grown selectively on a selection medium according to per se known methods. Corresponding clones are obtained from colonies. For analysis and further use, the sequences of the specific molecules can be determined according to conventional methods, thereby determining the variances, and the biological fine characterization of the specific molecules in the form of specific molecules expressed in bacteria or in the form of fusion proteins on phages, produced using superinfection by means of a helper phage, can be investigated in suitable test systems.

For example, the inactivation of phages failing to express the specific molecule can be effected using a helper phage which is inactivated by an inactivating substance. Thus, for example, a helper phage bearing a pIII can be used which, in contrast to the pIII of the fusion protein, can be cleaved by proteolysis, e.g. by trypsin in the event of a helper phage modified with a trypsin cleavage site in the pIII.

Optionally, the steps 1-3 can be performed repeatedly in further selection cycles. To this end, the bacteria infected with the eluted phages are made to produce phages including the specific molecules by means of a superinfection with a helper phage. Due to the phagemid system, a majority of the phages produced do not bear any specific molecule—as is the case when obtaining the phages of the overall library.

As set forth above, the sequence of the steps described can be changed such that the step now designated step 3 is performed prior to the step now designated step 1, i.e., prior to the incubation with target molecule, specific elution and respective wash steps. Being routine to those skilled in the art, the wash steps, which depend on the target molecule, selection molecule and specific molecules, will not be explained in detail. They can be inferred from the examples.

Surprisingly, large sets of specific molecules can be isolated with the method according to the invention, which exhibit an exceedingly high, heretofore unequalled diversity, and nevertheless have retained their ligand specificity. This is achieved by combining the specific elution using the selection molecule and the infection inactivation of those phages which are unexpectedly eluted in a non-specific fashion and do not bear any specific molecules. The method allows for isolation of a large variance of specific molecules after only one or a few selection cycles, with two or three selection cycles preferably being used.

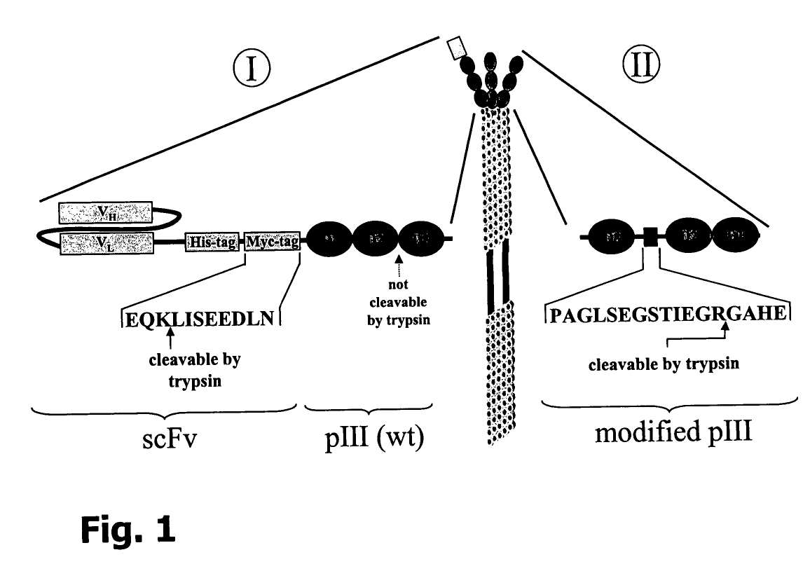

In a preferred embodiment of the invention, the method can be used to isolate large variances of surrogate molecules for a tumor-associated antigen (e.g. Lewis Y) in the form of antibody fragments such as scFv, single-domain and Fab antibody fragments, using specific antibodies. To this end, scFv-pIII (wild type) phagemid vectors and modified pIII helper phages rendered susceptible to proteolytic cleavage by introducing a trypsin cleavage site are used. One example is illustrated in FIG. 1 (see also Example 1). The use of the helper phage capable of undergoing proteolytic cleavage in combination with the antigen-specific elution (e.g. with Lewis Y antigen) results in a surprisingly high variance of surrogate molecules in the form of scFv fragments which inhibit binding of the antibody to the antigen.

In another preferred embodiment of the invention, the method can be used to isolate large variances of surrogate molecules in the form of antibody fragments such as scFv, single-domain and Fab antibody fragments which are directed against receptors (e.g. ELAM-1 selectin) and represent surrogate molecules for the ligands thereof (e.g. sialyl-Lewis A). To this end, scFv-pIII (wild type) phagemid vectors and modified pIII helper phages rendered susceptible to proteolytic cleavage by introducing a trypsin cleavage site are used. One representative example is illustrated in Example 2. The use of the helper phage capable of undergoing proteolytic cleavage in combination with the antigen-specific elution by the ligand or portions of the ligand (e.g. sialyl-Lewis A antigen) results in a surprisingly high variance of surrogate molecules in the form of scFv fragments which inhibit binding of receptor and ligand. For example, the ligands and the surrogate molecules thereof may act as agonists or antagonists for certain biological processes.

In another preferred embodiment of the invention, the method can be used to isolate variances of specific molecules having a particularly high affinity to the target molecule. For example, this can be achieved by effecting a pre-elution step with the selection molecule prior to the actual specific elution, which step elutes specific molecules having weaker binding so that only those specific molecules having more significant or high affinity will be isolated by the subsequent specific elution. For example, the pre-elution step can be accomplished by using a lower concentration of selection molecule or by using another molecule having weaker binding to the target molecule at the desired position as compared to the selection molecule. Alternatively, lower concentrations of selection molecule, or another molecule having weaker binding to the target molecule at the desired position than the selection molecule can be employed competitively in binding of the phages to the target molecule, and specific elution with the selection molecule can be performed as described. This is described in more detail in Example 3 wherein larger variances of higher-affinity surrogate molecules for the Lewis Y antigen in the form of antibody fragments are isolated by using an antibody which, in addition to Lewis Y, has lower affinity to Lewis B, and using Lewis B in the pre-elution and Lewis Y in the elution. To this end, scFv pIII (wild type) phagemid vectors and modified pIII helper phages rendered susceptible to proteolytic cleavage by introducing a trypsin cleavage site are used. The use of the helper phage capable of undergoing proteolytic cleavage in combination with the antigen-specific elution (e.g. with Lewis Y antigen) and pre-elution with a smaller amount of an alternative antigen (e.g. Lewis B) having weaker binding to the antibody results in a surprisingly high variance of higheraffinity surrogate molecules in the form of scFv fragments which exhibit particularly strong inhibition of binding of the antibody to the antigen.

Apart from the selection method, the present invention is also directed to a test kit for performing the inventive method, which test kit includes the components required therefor. In one embodiment of the invention, the test kit may include the phagemid gene library and a helper phage which can be inactivated by means of an inactivating substance. Given the special embodiment of proteolytic phage inactivation, for example, the test kit thus may include a helper phage with protease-sensitive pIII. In addition, the test kit may comprise further components such as the inactivating agent and preferably one or more proteases, with trypsin being particularly preferred. Furthermore, the test kit may include one or more target molecules and/or one or more selection molecules.

In another embodiment of the invention, the test kit used to perform the method of the invention includes at least one or more target molecules and one or more selection molecules as described above to perform the method.

Without intending to be limiting, the invention will now be described in more detail with reference to the examples.

### EXAMPLE 1

Generation of Surrogate Molecules Based on Antibody Fragments for the Lewis Y Carbohydrate Structure and Comparison of Different Elution Methods

The Lewis Y antibody which recognizes the tetrasaccharide Lewis Y was obtained as a cell culture supernatant from the hybridoma A70-C/C8 (IgM, K) according to standard methods and used in the selection method as target molecule to generate Lewis Y surrogate molecules.

A synthetic phagemid antibody library obtained according to well-known standard methods (Nissim, A. et al. 1994, EMBO J. 13, 692-69; Griffiths, A. et al., 1994, EMBO J. 13, 3245-3260; http://www.mrc-cpe.cam.ac.uk/˜phage/) was used in the investigations. The main characteristics of the library are the following: It is based on one single human framework for the heavy chain V_{H} (V3-23/DP-47 and J_{H}4b) and light chain V_{L} (O12/O2/DPK9 and J_{L}1), which represents the most frequent human canonical structure; the CDR3 region of the heavy chain has the minimum length required to form an antigen binding site; in the CDR2 and CDR3 regions, those amino acids were varied which make contact to the antigen and exhibit highest diversity in the natural, matured repertoire; the phagemid vector is based on the well-known pHEN vector; the library was subjected to a pre-selection for properly folded molecules by binding to protein L and protein A and has a diversity of 0.5-2.5×10⁸. The protease-sensitive helper phage KM13 was used as helper phage (Kristensen and Winter, 1998). The principle is described in FIG. 1 which shows the structure of a phage bearing an exemplary fusion protein molecule of scFv with non-modified pIII and other protease-sensitive, modified pIII molecules encoded by the genetic material of the helper phage. During packaging of the phages of a phagemid library induced by superinfection with helper phages, the modified pIII of the helper phage (FIG. 1, II) and the scFv pIII fusion protein (FIG. 1, I) compete for incorporation in the phage particle. KM13 encodes a modified pIII with additional protease cleavage sites, including a trypsin cleavage site between the domains D2 and D3 (FIG. 1, II). All of the three domains of pIII are essential for phage infectivity (Riechmann and Holliger, 1997), and it is possible to incorporate peptides between the domain boundaries without destroying the infectivity (Smith, 1985, Krabber et al., 1997). The wild type pIII (pIII(wt)) of the scFv-pIII(wt) fusion protein (FIG. 1, I) cannot be cleaved by trypsin, thereby receiving the infectivity of the phage particle following trypsin cleavage. About 99% of the phages produced in the phagemid system are void of scFv and will lose their infectivity by trypsin treatment. The trypsin cleavage site in the Myc-tag of the scFv-pIII(wt) allows for direct proteolytic elution of an infectious phage from the antigen, which was utilized in the 5^{th} elution method tested (see below). Production and determination of the helper phage titer were performed according to per se known methods (Kristensen and Winter, 1998).

Phage production from the bacterial library or from subsequent selections was induced by means of a superinfection with the KM13 helper phage. To this end, bacterial cultures in the stage of logarithmic growth (OD₆₀₀=0.5-0.6) were infected with the helper phages at a ratio of 1:20 (number of bacteria/number of phage particles) for 30 min at 37° C. and cultured in selection medium (TY including 100 μg/ml ampicillin and 25 μg/ml kanamycin) at 30° C. overnight. The phage particles were obtained from the culture supernatant by polyethylene glycol precipitation and resuspended in PBS.

The phage titer (number of phage particles/ml) was determined using dilution series of the phage preparations and subsequent infection of E. coli TG1. The amount of scFv-bearing phage particles was determined using comparative titration of trypsinated and non-trypsinated phage preparations.

In the selections, the cell culture supernatant of the A70-C/C8 hybridoma was immobilized on mouse IgM-specific magnetic particles (Dynal, Hamburg; 6 ml supernatant/200 μl magnetic particles, at least 2 hours at room temperature (RT)). Following a wash step using TPBS (PBS/0.1% Tween20), the magnetic particles coupled to A70-C/C8 were blocked with 4% MPBS (4% skim milk powder in PBS) for 1 hour at RT. In the first selection cycle, 5×10¹² phage particles statistically including about 500 copies of each library scFv were used and incubated with the A70-C/C8-bearing magnetic balls for 2 hours at RT. Subsequently, the magnetic balls were washed (first selection cycle: 8×2% MPBS+8×PBS/0.1% Tween20+2×PBS; further selection cycles: 16×2% MPBS+16×PBS/0.1% Tween20+2×PBS), and the phages were eluted.

For phage elution, miscellaneous elution methods were used in comparison:

1. non-specific elution

2. non-specific elution with subsequent trypsination

3. specific elution

4. specific elution with subsequent trypsination

5. elution by trypsination (enabled by a trypsin cleavage site in Myc-tag; FIG. 1)

In non-specific elution, 50 mM glycine buffer (pH 2.2; 450 μl) was used to remove the phages from the magnetic particles (30 min at RT).

For specific elution, a PAA-coupled Lewis Y tetrasaccharide was used as antigen (450 μl, 100 μg/ml in 50 mM Tris, 1 mM CaCl₂′ pH 8).

Trypsination was effected at a concentration of 1 mg/ml in 500 μl 50 mM Tris, 1 mM CaCl₂₁ pH 8, for 15 min at RT.

The eluted phages were used to infect 9.5 ml E. coli TG1 in their logarithmic phase of growth (30 min 37° C.), which then were plated on 2×TY plates together with ampicillin and glucose, and the titer and thus, the amount of eluted phages was determined by means of dilution series. For further selection cycles, the colonies were isolated from the plates and preserved as a glycerol permanent culture (2×TY/15-20% glycerol) at −80° C. These permanent cultures (100 μl) were used for initial growth of bacteria in the next selection cycle. Single colonies were picked for further analysis, frozen permanent cultures were prepared, and phage supernatants were produced which then were analyzed for specific anti-idiotypic binding using ELISA.

The phage supernatants were produced by superinfection with KM13 and PEG precipitation as described in the library phage preparation. This was done in miscellaneous volume formats as required. For ELISA specificity testing, the phages were furnished in 200 μl volumes in 96-well culture plates. The phages used in the phage inhibition tests were obtained from 120 ml supernatant using repeated PEG precipitation in 2 ml PBS and concentrated.

For the ELISA specificity test, a polyclonal p chain-specific goat-anti-mouse IgM antibody (anti-IgM) was immobilized on a 96-well plate (240 ng/well, 4° C. overnight, on Maxisorb, Nunc), which antibody, following washing (TPBS) and blocking of the plate (4% MPBS), resulted in binding of the specific antibody (A70-C/C8 supernatant, 1:3 diluted hybridoma culture supernatant) or a control antibody of the same isotype (TEPC183). Equal amounts for use were adjusted via the same antibodies in purified form. Subsequently, this was incubated with the PEG-precipitated scFv phages of the individual clones. Binding of the scFv phages was detected using a monoclonal peroxidase-coupled anti-M13-antibody (Amersham Pharmacia Biotech, Freiburg, Germany). 3′3′5′5′-tetramethylbenzidine was used as substrate, and the staining reaction was quenched by adding 2.5 N sulfuric acid. Measurement was effected at 450 and 620 nm.

Determination of binding to unoccupied, blocked surfaces (binding to plastic or blocking agent) and to polyclonal μ chain-specific goat-anti-mouse IgM antibody (trans-species Ig binder) was used as additional control. All of the washing procedures between the individual incubation steps were effected using TPBS (4×), with 2 additional PBS wash cycles prior to addition of substrate.

Binding to A70-C/C8 with an absorption of at least 0.15 (=A₄₅₀−A₆₂₀) and binding to the control antibodies with less than 20% of binding to A70-C/C8 (each time after subtracting the background values) were established as threshold values for the identification of scFv phages, which represent the selection criteria for surrogate molecules for Lewis Y in the ELISA. For an isotype binder, the signal for binding to A70-C/C8 and to TEPC183 should have an OD of more than 0.15 at a background signal of <50%. General antibody binders were defined by their binding to A70-C/C8, TEPC183 and anti-IgM, and non-specific binders by binding with no immobilized antibodies.

Clones complying with the criteria of a surrogate molecule—binding to the LeY-specific antibody and no binding to controls—were subsequently subjected to sequencing. Sequencing was effected with standard methods in such a way that the V_{H} and V_{L} genes of the isolated phages were amplified by PCR using Taq polymerase and the primers CAG GAA ACA GCT ATG AC and GAA TTT TCT GTA TGA GG with 25 cycles from bacteria frozen cultures (20% glycerol) of the clones, and the products were purified with QIAquick (PCR purification kit, QIAgen) and sequenced using the primer CTA TGC GGC CCC ATT CA. Sequencing was effected with the aid of fluorescent dideoxy chain terminators (Dye Terminator Sequencing Kit II, Amersham Pharmacia Biotech, Freiburg, Germany) according to a standard method (GAG, Bremen, Germany).

Clones of varying sequences were tested in inhibition analyses in an ELISA for their inhibitory effect as surrogate molecules of the antigen. To this end, 10 μg/ml Lewis Y PAA in PBS was immobilized for 3 hours at RT and at 4° C. overnight. The plates were washed and blocked with 4% MPBS for 2 hours at RT. A70-C/C8 hybridoma culture supernatant (1:5 and 1:20) was pre-incubated with varying amounts of scFv phages (1012 to 1010, about 1% of which bearing scFv) in 2% MPBS/0.1% Tween20 for 1 hour at RT in a blocked tube. Subsequently, 50 μl/well was placed on the antigen-coated plate for 1.5 hours at RT. Thereafter, this was washed four times with PBS/0.1% Tween20 and incubated with the secondary antibody (peroxidase-conjugated rabbit-anti-mouse immunoglobulin, 1:2000) in 2% MPBS/0.1% Tween20 for 1 hour at RT. The plates were washed and developed as described in the above ELISA.

Table 1 presents a comparative illustration of the effect of miscellaneous elution techniques. Therein, surrogate scFv are those binding to A70-C/C8 only, but not to the controls. Isotype binders are those binding to A70-C/C8 and to antibodies of the same isotype (mouse IgM). General antibody binders are those binding to all of the antibodies used. Non-specific binders bind to the plate material or to the blocking agent. Non-binding phages are those failing to bind.

Table 2 shows the yield of surrogate molecules obtained according to the method of the invention after 2 and 3 selection cycles using specific elution with subsequent trypsination and their diversity determined by sequencing.

The results show that the selection method of the invention with combined elution and subsequent trypsination is far superior to other well-known and tested elution methods of generating high variances.

According to the invention, 28 surrogate molecules were isolated in the form of scFv antibody fragments for LeY from 96 clones after only 2 selection cycles. An additional selection cycle resulted in 76 surrogate molecule clones from 96 tested clones. Among these surrogate molecules, 65-67% had different amino acid sequences and surprisingly, no significant difference in the variance was noted when using 2 vs. 3 selection cycles. Massive accumulation of phages including specific surrogate molecules was accompanied by a depletion of non-specific scFv phages and those scFv phages including general antibody binders. In contrast, the other elution methods accumulated general antibody binders, non-specific binders and non-binders, achieving no accumulation of surrogate scFv or, in the event of specific elution alone, very low, negligible accumulation thereof.

Inhibition tests in an ELISA demonstrate that virtually all of the isolated surrogate scFv specifically inhibit binding of Lewis Y antigen to A70-C/C8.

### EXAMPLE 2

Generation of Surrogate Molecules Based on Antibody Fragments for the Carbohydrate Ligand of ELAM-1 Selectin

The cytoplasmatic domain of ELAM-1 selectin was used in recombinant form (from CHO cells, R+D Systems #ADP-1) in the tests. In the presence of Ca²⁺, ELAM-1 selectin binds the sialyl-Lewis A and sialyl-Lewis X ligands and is of importance in cell adhesion e.g. of activated leukocytes to the endothelium.

A synthetic phagemid antibody library obtained according to well-known standard methods (Nissim, A. et al. 1994, EMBO J. 13, 692-69; Griffiths, A. et al., 1994, EMBO J. 13, 3245-3260; http://www.mrc-cpe.cam.ac.uk/˜phage/) was used in the investigations. The main characteristics of the library are the following: It is based on one single human framework for the heavy chain V_{H} (V3-23/DP-47 and J_{H}4b) and light chain V_{L} (O12/O2/DPK9 and J_{L}1), which represents the most frequent human canonical structure; the CDR3 Region of the heavy chain has the minimum length required to form an antigen binding site; in the CDR2 and CDR3 regions, those amino acids were varied which make contact to the antigen and exhibit highest diversity in the natural, matured repertoire; the phagemid vector is based on the well-known pHEN vector; the library was subjected to a pre-selection for properly folded molecules by binding to protein L and protein A and has a diversity of 0.5-2.5×10⁸.

The protease-sensitive helper phage KM13 was used as helper phage (Kristensen and Winter, 1998). The principle is described in FIG. 1 which shows the structure of a phage bearing an exemplary fusion protein molecule of scFv with non-modified pIII and other protease-sensitive, modified pIII molecules encoded by the genetic material of the helper phage. During packaging of the phages of a phagemid library induced by superinfection with helper phages, the modified pIII of the helper phage (FIG. 1, II) and the scFv pIII fusion protein (FIG. 1, I) compete for incorporation in the phage particle. KM13 encodes a modified pIII with additional protease cleavage sites, including a trypsin cleavage site between the domains D2 and D3 (FIG. 1, II). All of the three domains of pIII are essential for phage infectivity (Riechmann and Holliger, 1997), and it is possible to incorporate peptides between the domain boundaries without destroying the infectivity (Smith, 1985, Krabber et al., 1997). The wild type pIII (pIII(wt)) of the scFv-pIII(wt) fusion protein (FIG. 1, I) cannot be cleaved by trypsin, thereby receiving the infectivity of the phage particle following trypsin cleavage. About 99% of the phages produced in the phagemid system are void of scFv and will lose their infectivity by trypsin treatment. Production and determination of the helper phage titer were performed according to per se known methods (Kristensen and Winter, 1998).

Phage production from the bacterial library or from subsequent selections was induced by means of a superinfection with the KM13 helper phage. To this end, bacterial cultures in the stage of logarithmic growth (OD₆₀₀=0.5-0.6) were infected with the helper phages at a ratio of 1:20 (number of bacteria/number of phage particles) for 30 min at 37° C. and cultured in selection medium (TY including 100 μg/ml ampicillin and 25 μg/ml kanamycin) at 30° C. overnight. The phage particles were obtained from the culture supernatant by polyethylene glycol precipitation and resuspended in PBS.

The phage titer (number of phage particles/ml) was determined using dilution series of the phage preparations and subsequent infection of E. coli TG1.

In the selections, 60 μg of recombinant ELAM-1 selectin in 2 ml was immobilized on immunotubes overnight at 4° C. in TC buffer (50 mM Tris, pH 7.4, 10 mM CaCl₂). Following a wash step with TC buffer (3×1 volume), the immunotube was blocked with 30% FKS/RPMI/TC and the phage library with 10% FKS/RPMI/TC for 1.5 hours at RT. In the first selection cycle, 5×10¹² phage particles statistically including about 500 copies of each library scFv were used and incubated with the phagemid scFv library for 0.5 hours at RT. Subsequently, the immunotube was washed (first selection cycle: 4×10% FKS/RPMI/TC+4×PBS/0.1% Tween20+2×PBS; further selection cycles: 5×10% FKS/RPMI/TC+6×TC/0.1% Tween20+4×TC). Thereafter, the phages were specifically eluted with pAA-coupled sialyl-Lewis A tetrasaccharide (450 μl, 111 μg/ml in 50 mM Tris, 1 mM CaCl₂, pH 8), and the eluted phages were treated with trypsin for 15 min at RT (1 mg/ml trypsin in 500 μl of 50 mM Tris, 1 mM CaCl₂, pH 8).

The eluted phages were used to infect 9.5 ml E. coli TG1 in their logarithmic phase of growth (30 min 37° C.), which then were plated on 2×TY plates together with ampicillin and glucose, and the titer and thus, the amount of eluted phages was determined by means of dilution series. For further selection cycles, the colonies were isolated from the plates and preserved as a glycerol permanent culture (2×TY/15-20% glycerol) at −80° C. These permanent cultures (100 μl) were used for initial growth of bacteria in the next selection cycle. Single colonies were picked for further analysis, frozen permanent cultures were prepared, and phage supernatants were produced which then were analyzed for specific anti-idiotypic binding using ELISA.

The phage supernatants were produced by superinfection with KM13 and PEG precipitation as described in the library phage preparation. This was done in miscellaneous volume formats as required. For ELISA specificity testing, the phages were furnished in 200 μl volumes in 96-well culture plates. The phages used in the phage inhibition tests were obtained from 120 ml supernatant using repeated PEG precipitation in 2 ml PBS and concentrated.

For the ELISA specificity test, ELAM-1 selectin in TC buffer (50 mM Tris, pH 7.4, 10 mM CaCl₂) was immobilized on a 96-well plate (200 ng/well, 4° C. overnight, pvc falcon) and, following washing (TC) and blocking of the plate (30% FKS/RPMI/TC), used to bind the specific phage. Subsequently, this was incubated with the PEG-precipitated scFv phages (about 10¹²/ml) of the individual clones. Binding of the scFv phages was detected using a monoclonal peroxidase-coupled anti-M13-antibody (Amersham Pharmacia Biotech, Freiburg, Germany). 3′3′5′5′-tetramethylbenzidine was used as substrate, and the staining reaction was quenched by adding 2.5 N sulfuric acid. Measurement was effected at 450 and 620 nm.

Determination of binding to unoccupied, blocked surfaces (binding to plastic or blocking agent) was used as additional control. All of the washing procedures between the individual incubation steps were effected using TC/0.1% Tween (4 times), with 2 additional TC wash cycles prior to addition of substrate.

Binding to ELAM-1 selectin with an absorption of at least 0.15 (=A₄₅₀−A₆₂₀) and binding in the control wells with less than 20% of binding to ELAM-1 selectin (each time after subtracting the background values) were established as threshold values for the identification of scFv phages, which represent the selection criteria for surrogate molecules for sialyl-Lewis A in the ELISA.

Clones complying with the criteria of a surrogate molecule for the ELAM-1 selectin ligand were subsequently subjected to sequencing. Sequencing was effected with standard methods in such a way that the V_{H} and V_{L} genes of the isolated phages were amplified by PCR using Taq polymerase and the primers CAG GAA ACA GCT ATG AC and GAA TTT TCT GTA TGA GG with 25 cycles from bacteria frozen cultures (15-20% glycerol) of the clones, and the products were purified with QIAquick (PCR purification kit, QIAgen) and sequenced using the primer CTA TGC GGC CCC ATT CA. Sequencing was effected with the aid of fluorescent dideoxy chain terminators (Dye Terminator Sequencing Kit II, Amersham Pharmacia Biotech, Freiburg, Germany) according to a standard method (GAG, Bremen, Germany).

Clones of varying sequences were tested in inhibition analyses in an ELISA for their inhibitory effect as surrogate molecules of the antigen. To this end, ELAM-1 selectin in TC buffer (50 mM Tris, pH 7.4, 10 mM CaCl₂) was immobilized on a 96-well plate (200 ng/well, 4° C. overnight, pvc falcon) and, following washing (TC) and blocking of the plate (30% FKS/RPMI/TC), used to bind the specific sugar (sialyl-Lewis A-PAA-biotin and sialyl-Lewis A-PAA, respectively). Subsequently, incubation with the PEG-precipitated scFv phages of the individual clones was effected at varying concentrations of from 0 to about 1011 phages/well. Binding of the scFv phages or of sialyl-Lewis A-PAA-biotin was detected using a monoclonal peroxidase-coupled anti-M13-antibody (Amersham Pharmacia Biotech, Freiburg, Germany) and a peroxidase-coupled streptavidin. 3′3′5′5′-tetramethylbenzidine was used as substrate, and the staining reaction was quenched by adding 2.5 N sulfuric acid.

Measurement was effected at 450 and 620 nm. The plates were washed and developed as described in the above ELISA.

Table 3 shows the yield of surrogate molecules obtained after 2 selection cycles of the invention using specific elution with subsequent trypsination and their diversity determined by sequencing.

The results show that the selection method of the invention with combined elution and subsequent trypsination achieves high variance of surrogate ligands for the ELAM-1 selectin carbohydrate ligand.

Inhibition tests in an ELISA demonstrate that virtually all of the isolated surrogate scFv specifically inhibit binding of the sialyl-Lewis A antigen to the recombinant ELAM-1 selectin.

### EXAMPLE 3

Generation of High-Affinity Surrogate Molecules Based on Antibody Fragments for the Lewis Y Carbohydrate Structure

The antibody A70-A/A9 recognizes the tetrasaccharide Lewis Y and has a cross-reactivity with the tetrasaccharide Lewis B which is by about 20-100 times weaker than binding to Lewis Y. The mouse antibody was obtained as a cell culture supernatant from the hybridoma A70-A/A9 (IgG1, K) according to standard methods and used in the selection method as target molecule to generate Lewis Y surrogate molecules.

A synthetic phagemid antibody library obtained according to well-known standard methods (Nissim, A. et al. 1994, EMBO J. 13, 692-69; Griffiths, A. et al., 1994, EMBO J. 13, 3245-3260; http://www.mrc-cpe.cam.ac.uk/˜phage/) was used in the investigations. The main characteristics of the library are the following: It is based on one single human framework for the heavy chain V_{H} (V3-23/DP-47 and J_{H}4b) and light chain V_{L} (O12/O2/DPK9 and J_{L}1), which represents the most frequent human canonical structure; the CDR3 Region of the heavy chain has the minimum length required to form an antigen binding site; in the CDR2 and CDR3 regions, those amino acids were varied which make contact to the antigen and exhibit highest diversity in the natural, matured repertoire; the phagemid vector is based on the well-known pHEN vector; the library was subjected to a pre-selection for properly folded molecules by binding to protein L and protein A and has a diversity of 0.5-2.5×10⁸.

The protease-sensitive helper phage KM13 was used as helper phage (Kristensen and Winter, 1998). The principle is described in FIG. 1 which shows the structure of a phage bearing an exemplary fusion protein molecule of scFv with non-modified pIII and other protease-sensitive, modified pIII molecules encoded by the genetic material of the helper phage. During packaging of the phages of a phagemid library induced by superinfection with helper phages, the modified pIII of the helper phage (FIG. 1, II) and the scFv pIII fusion protein (FIG. 1, I) compete for incorporation in the phage particle. KM13 encodes a modified pIII with additional protease cleavage sites, including a trypsin cleavage site between the domains D2 and D3 (FIG. 1, II). All of the three domains of pIII are essential for phage infectivity (Riechmann and Holliger, 1997), and it is possible to incorporate peptides between the domain boundaries without destroy the infectivity (Smith, 1985, Krabber et al., 1997). The wild type pIII (pIII(wt)) of the scFv-pIII(wt) fusion protein (FIG. 1, I) cannot be cleaved by trypsin, thereby receiving the infectivity of the phage particle following trypsin cleavage. About 99% of the phages produced in the phagemid system are void of scFv and will lose their infectivity by trypsin treatment. The trypsin cleavage site in the Myc-tag of the scFv-pIII(wt) allows for direct proteolytic elution of an infectious phage from the antigen, which was utilized in the 5^{th} elution method tested (see below). Production and determination of the helper phage titer were performed according to per se known methods (Kristensen and Winter, 1998).

Phage production from the bacterial library or from subsequent selections was induced by means of a superinfection with the KM13 helper phage. To this end, bacterial cultures in the stage of logarithmic growth (OD₆₀₀=0.5-0.6) were infected with the helper phages at a ratio of 1:20 (number of bacteria/number of phage particles) for 30 min at 37° C. and cultured in selection medium (TY including 100 μg/ml ampicillin and 25 μg/ml kanamycin) at 30° C. overnight. The phage particles were obtained from the culture supernatant by polyethylene glycol precipitation and resuspended in PBS.

The phage titer (number of phage particles/ml) was determined using dilution series of the phage preparations and subsequent infection of E. coli TG1. The amount of scFv-bearing phage particles was determined using comparative titration of trypsinated and non-trypsinated phage preparations. In the selections, the cell culture supernatant of the A70-A/A9 hybridoma was immobilized on mouse IgG-specific magnetic particles (Dynal, Hamburg; 6 ml supernatant/200 μl magnetic particles, at least 2 hours at room temperature (RT)). Following a wash step using TPBS (PBS/0.1% Tween20), the magnetic particles coupled with A70-A/A9 were blocked with 4% MPBS (4% skim milk powder in PBS) for 1 hour at RT. In the first selection cycle, 5×10¹² phage particles statistically including about 500 copies of each library scFv were used and incubated with the A70-A/A9-bearing magnetic balls for 2 hours at RT. Subsequently, the magnetic balls were washed (first selection cycle: 8×2% MPBS+8×PBS/0.1% Tween20+2×PBS; further selection cycles: 16×2% MPBS+16×PBS/0.1% Tween20+2×PBS). Thereafter, the phages with pAA-coupled Lewis B tetrasaccharide (450 μl, 5 μg/ml in 50 mM Tris, 1 mM CaCl₂, pH 8) were eluted in a pre-elution step. Subsequently, this was washed twice with PBS, and the actual elution was performed with pAA-coupled Lewis Y tetrasaccharide (450 μl, 100 μg/ml in 50 mM Tris, 1 mM CaCl₂, pH 8). The eluted and pre-eluted phages were treated separately with trypsin (1 mg/ml trypsin in 500 μl of 50 mM Tris, 1 mM CaCl₂₁ pH 8, for 15 min at RT).

The eluted and pre-eluted phages were used to infect 9.5 ml E. coli TG1 in their logarithmic phase of growth (30 min 37° C.), which then were plated on 2xTY plates together with ampicillin and glucose, and the titer and thus, the amount of eluted phages was determined by means of dilution series. For further selection cycles, the colonies were isolated from the plates and preserved as a glycerol permanent culture (2×TY/15-20% glycerol) at −80° C. These permanent cultures (100 μl) were used for initial growth of bacteria in the next selection cycle. Single colonies were picked for further analysis, frozen permanent cultures were prepared, and phage supernatants were produced which then were analyzed for specific anti-idiotypic binding using ELISA.

The phage supernatants were produced by superinfection with KM13 and PEG precipitation as described in the library phage preparation. This was done in miscellaneous volume formats as required. For ELISA specificity testing, the phages were furnished in 200 μl volumes in 96-well culture plates. The phages used in the comparative relative affinity tests were obtained from 40 ml supernatant using PEG precipitation in 2 ml PBS and concentrated.

For the ELISA specificity test, a polyclonal γ chain-specific goat-anti-mouse IgG antibody (anti-IgG) was immobilized on a 96-well plate (240 ng/well, 4° C. overnight, on Maxisorb, Nunc), which antibody, following washing (TPBS) and blocking of the plate (4% MPBS), resulted in binding of the specific antibody (A70-A/A9 supernatant, 1:3 diluted hybridoma culture supernatant) or a control antibody of the same isotype (MOPC-21). Equal amounts for use were adjusted via the same antibodies in purified form. Subsequently, this was incubated with the PEG-precipitated scFv phages of the individual clones. Binding of the scFv phages was detected using a monoclonal peroxidase-coupled anti-M13-antibody (Amersham Pharmacia Biotech, Freiburg, Germany). 3′3′5′5′-tetramethylbenzidine was used as substrate, and the staining reaction was quenched by adding 2.5 N sulfuric acid. Measurement was effected at 450 and 620 nm.

Determination of binding to unoccupied, blocked surfaces (binding to plastic or blocking agent) and to polyclonal μ chain-specific goat-anti-mouse IgM antibody (trans-species Ig binder) was used as additional control. All of the washing procedures between the individual incubation steps were effected using TPBS (4×), with 2 additional PBS wash cycles prior to addition of substrate.

Binding to A70-A/A9 with an absorption of at least 0.15 (=A₄₅₀−A₆₂₀) and binding to the control antibodies with less than 20% of binding to A70-A/A9 (each time after subtracting the background values) were established as threshold values for the identification of scFv phages, which represent the selection criteria for surrogate molecules for Lewis Y in the ELISA. For an isotype binder, the signal for binding to A70-A/A9 and to MOPC-21 should have an OD of more than 0.15 at a background signal of <50%. General antibody binders were defined by their binding to A70-A/A9, MOPC-21 and anti-IgG, and non-specific binders by binding with no immobilized antibodies.

Clones complying with the criteria of a surrogate molecule—binding to A70-A/A9 and no binding to controls—were tested for their relative affinity to A70-A/A9. The titers of the PEG-precipitated and concentrated phages were determined, and equal phage concentrations were subjected to comparative testing in dilution series in an ELISA with A70-A/A9 as described above.

Phage preparations of clones complying with the criteria of a surrogate molecule were subsequently subjected to sequencing.

Sequencing was effected with standard methods in such a way that the V_{H} and V_{L} genes of the isolated phages were amplified by PCR using Taq polymerase and the primers CAG GAA ACA GCT ATG AC and GAA TTT TCT GTA TGA GG with 25 cycles from bacteria frozen cultures (15-20% glycerol) of the clones, and the products were purified with QIAquick (PCR purification kit, QIAgen) and sequenced using the primer CTA TGC GGC CCC ATT CA. Sequencing was effected with the aid of fluorescent dideoxy chain terminators (Dye Terminator Sequencing Kit II, Amersham Pharmacia Biotech, Freiburg, Germany) according to a standard method (GAG, Bremen, Germany).

Table 4 shows that both in pre-elution and in the subsequent elution, nearly comparable amounts of phages were eluted after the 2^{nd} selection cycle, both having high variance.

A comparison of 8 clones from the pre-elution and from the elution after the 2^{nd} selection cycle shows that the isolated surrogate molecules from the elution have an affinity to A70-A/A9 ranging from significantly to many times higher compared to the surrogate molecules from the pre-elution.

TABLE 1

| | | | Non-specific | | | | | | | |
|---|---|---|---|---|---|---|---|---|---|---|
| | | | elution | | | | Specific elution | | | |
| | Non-specific | | with subsequent | | | | with subsequent | | Elution by trypsin | |
| | elution | | trypsination | | Specific elution | | trypsination | | treatment | |
| Selection | 1 | 2 | 2 | 1 | 1 | 2 | 1 | 2 | 1 | 2 |
| cycles | | | | | | | | | | |
| Total number | 2 × 10⁶ | 6 × 10⁵ | 9.4 × 10⁴ | 6 × 10⁵ | 6 × 10⁴ | 2.5 × 10³ | 1.3 × 10³ | 1.2 × 10³ | 2.6 × 10³ | 2.7 × 10⁴ |
| of colonies | | | | | | | | | | |

Number of binding clones out of 96 clones tested in the ELISA

| | | | | | | | | | | |
|---|---|---|---|---|---|---|---|---|---|---|
| Surrogate | 5 | 1 | 1 | 0 | 0 | 2 | 3 | 28 | 9 | 1 |
| scFv | | | | | | | | | | |
| Isotype binder | 1 | 0 | 1 | 0 | 0 | 0 | 0 | 1 | 0 | 0 |
| General | 33 | 15 | 35 | 29 | 25 | 16 | 33 | 24 | 28 | 49 |
| antibody | | | | | | | | | | |
| binder | | | | | | | | | | |
| Non-specific | 2 | 3 | 0 | 3 | 3 | 5 | 1 | 1 | 1 | 0 |
| binder | | | | | | | | | | |
| Non-binding | 55 | 77 | 59 | 64 | 68 | 73 | 59 | 42 | 58 | 46 |
| clones | | | | | | | | | | |

TABLE 2

| | |
|---|---|
| | +TC,16/31 A70-C/C8 |
| Selection molecule | LeY-PAA [100 μg/ml] |

| | | |
|---|---|---|
| Selection cycles | 2 | 3 |
| Selection with | Magnetic balls | Magnetic balls |

Number of binding clones out of 96 clones tested in the ELISA

| | | |
|---|---|---|
| Surrogate scFv | 28 | 76 |
| Isotype binder | 1 | 0 |
| Non-specific binder | 1 | 0 |
| Non-binding clones | 42 | 11 |
| Diversity | | |
| Number of sequenced clones | 23 | 18 |
| Number of varying sequences | 15 | 12 |
| Diversity in % | 65 | 67 |

TABLE 3

| | |
|---|---|
| Target molecule | ELAM-1 selectin |
| Selection molecule | Sialyl-LeA-PAA [111 μg/ml] |
| Selection cycles | 2 |
| Selection with | Magnetic balls |

Number of binding clones out of 96 clones tested in the ELISA

| | |
|---|---|
| Surrogate scFv | 37 |
| Non-specific binders | 8 |
| Non-binding clones | 51 |
| Number of sequenced clones | 12 |
| Number of varying sequences | 9 |
| Diversity in % | 75 |

TABLE 4
Target molecule A70-A/A9
Specific elution with subsequent trypsin treatment

| | Pre-elution | Elution |
|---|---|---|
| Selection molecule | LeB-PAA [5 μg/ml] | LeY-PAA [100 μg/ml] |
| Selection cycle | 2 | 2 |
| Total number of colonies | 2.3 × 10³ | 9 × 10² |

Number of binding clones out of 96 clones tested in the ELISA

| | | |
|---|---|---|
| Surrogate scFv | 39 | 32 |
| Isotype binder | 0 | 1 |
| Non-specific binder | 1 | 1 |
| Non-binding clones | 27 | 44 |
| Diversity | | |
| Number of sequenced | 16 | 16 |
| clones | | |
| Number of varying | 12 | 9 |
| sequences | | |
| Diversity in % | 75 | 56 |

1. A method for the isolation of large variances of specific molecules for a target molecule from phagemid gene libraries, said method being performed by starting with a phagemid gene library including large variances of a molecule or large variances of portions of a molecule which acts as a specific molecule, directly or via a linker, coupled covalently to the phage coat protein pIII of the filamentous phage M13 as a fusion protein on the surface of phages, incubating the library phages including the phages expressing the fusion protein with a target molecule, eluting the phages specifically bound to the target molecule via the fusion protein with a selection molecule which terminates binding between the specific molecule and the target molecule and binds to the target molecule, inactivating the eluted phages failing to express the fusion protein in a way so as to render them incapable of infecting bacteria, using the eluted phages expressing the fusion protein to infect bacteria, and determining and obtaining the variances of the specific molecule from the infected clones.

2. The method according to claim 1, characterized in that
carrier proteins including one or more variable portions, or antibody fragments, preferably Fab fragments, single-domain fragments or scFv fragments are isolated as specific molecules.

3. The method according to claim 1, characterized in that
those molecules are used as target molecules which represent receptors or portions thereof acting as receptors for the selection molecule, and as selection molecules, those are used which represent ligands or portions thereof acting as ligands for the target molecule.

4. The method according to claim 1, characterized in that
antibodies or fragments thereof specifically binding to the selection molecule are used as target molecules, and the antigens or portions thereof are used as selection molecules.

5. The method according to claim 1, characterized in that
lectins specifically binding to the selection molecule are used as target molecule, and carbohydrate structures or portions thereof are used as selection molecule.

6. The method according to claim 1, characterized in that
as specific molecules, those are isolated which represent surrogate molecules for the selection molecule or portions of the selection molecule.

7. The method according to claim 6, characterized in that
antibody fragments or carrier proteins are isolated as surrogate molecules, a receptor or a portion of a receptor is used as target molecule, which receptor binds to the ligand serving as selection molecule or to a portion of said ligand.

8. The method according to claim 6, characterized in that
antibody fragments are isolated as surrogate molecules, and an antibody or antibody fragment directed against the antigen serving as selection molecule or portions thereof is used as target molecule.

9. The method according to claim 1, characterized in that
the inactivation of phages failing to express the fusion protein and thus, the specific molecule, is effected by proteolysis using a helper phage including protease-sensitive pIII.

10. The method according to claim 1 for the isolation of large variances of surrogate molecules for antigens in the form of single-domain or scFv antibody fragments directed against antibodies recognizing a tumor-associated antigen,
characterized in that
as phagemid gene library, one is used which includes the antibody fragment pIII(wild type) phagemid vectors, antibodies or antibody fragments against the tumor-associated antigen are used as target molecule, the tumor-associated antigen or portions thereof are used as selection molecule, and the inactivation of phages failing to express the fusion protein is effected by proteolysis using a pIII helper phage which, as a result of modification, can be cleaved by proteolysis.

11. The method according to claim 1 for the isolation of large variances of surrogate molecules for ligands in the form of single-domain or scFv antibody fragments directed against receptors bound by a ligand, characterized in that
as phagemid gene library, one is used which includes the antibody fragment pIII(wild type) phagemid vectors, the receptor or portions of the receptor are used as target molecule, the ligand or portions of the ligand are used as selection molecule, and the inactivation of phages failing to express the fusion protein is effected by proteolysis using a pIII helper phage which, as a result of modification, can be cleaved by proteolysis.

12. The method according to claim 1, characterized in that
variances of specific molecules having higher affinity to the target molecule are obtained by performing an additional step with the selection molecule prior to specific elution, which step is a pre-elution wherein weaker binding specific molecules are pre-eluted with weaker binding molecules or with a lower concentration of selection molecule or a combination of both.

13. The method according to claim 1, characterized in that
variances of specific molecules having higher affinity to the target molecule are obtained by competitive incubation of phages bearing the specific molecules together with the target molecule at a lower concentration of selection molecule or of another molecule having weaker specific binding to the target molecule than the selection molecule, or a combination of both.

14. The method according to claim 1, characterized in that
the inactivation of phages failing to express the fusion protein is effected prior to incubation with target molecule and specific elution with selection molecule.

15. A test kit for the isolation of large variances of specific molecules for a target molecule from phagemid gene libraries according to the method of claims 1 to 14, comprising a phagemid gene library and a helper phage susceptible to inactivation.

16. The test kit according to claim 15, characterized in that
the kit comprises a helper phage including protease-sensitive pIII.

17. The test kit according to claim 15 or 16, characterized in that
the kit additionally comprises the agent for inactivating the helper phage, preferably one or more proteases.

18. The test kit according to any of claims 15 to 17, characterized in that
the kit comprises one or more target molecules and/or one or more selection molecules.

19. A test kit for the isolation of large variances of specific molecules for a target molecule from phagemid gene libraries according to the method of claims 1 to 14, comprising one or more target molecules and one or more selection molecules.
